(19) United States
(12) Patent Application Publication
(10) Pub. No.: US 2012/0004290 A1
(43) Pub. Date: Jan. 5, 2012

(54) TREATING CHRONIC FATIGUE SYNDROME AND PROLONGED QT INTERVAL

(76) Inventors: David Strayer, Bryn Mawr, PA (US); William A. Carter, Spring City, PA (US)

(21) Appl. No.: 13/256,039

(22) PCT Filed: Mar. 10, 2010
(86) PCT No.: PCT/US2010/000718

§ 371 Date: Sep. 12, 2011

Related U.S. Application Data
(60) Provisional application No. 61/160,290, filed on Mar. 13, 2009.

(51) Int. Cl. A61K 31/713; A61P 43/00; C07H 21/02

(52) U.S. Cl. 514/44 R; 536/23.1

(57) ABSTRACT

Chronic fatigue syndrome and prolonged QT interval are treated using one or more different double-stranded ribonucleic acids (dsRNA).

### FIELD OF THE INVENTION

The invention relates to the treatment of human patients having chronic fatigue syndrome and prolonged QT interval. Medicaments, processes for their manufacture, and methods for their use are provided herein.

### BACKGROUND OF THE INVENTION

Chronic fatigue syndrome is characterized by persistent and disabling fatigue of at least six months duration, which is not explained by another medical condition. See Afari & Buchwald, American Journal of Psychiatry, 160: 221-236 (2003). Many different drugs are administered to patients in an attempt to alleviate the severity of symptoms or reduce their number. Unfortunately, although approved for general use, many of these drugs have an associated product warning that exposure to them may result in prolonged QT interval, which translates into increased risk of cardiotoxicity. Other patients are affected by certain preconditions to a cardiac disorder associated with chronic fatigue syndrome itself.

Here, we show that treatment of patients having prolonged QT interval with a specifically-configured dsRNA normalizes their electrocardiograms and reduces their use of medications that result in prolonged QT interval.

U.S. Pat. No. 6,130,206 describes a method of treating patients suffering from chronic fatigue syndrome with dsRNA. This subset of patients had many different viruses replicating in them. They include cytomegalovirus, Epstein-Barr virus, other human herpes viruses, and retroviruses. It was discovered that the activity of 2′-5′ oligoadenylate synthetase was abnormally low and ribonuclease (RNase) L acquired aberrant new activities in lymphocytes from the subset of virally-infected patients.

U.S. Pat. No. 5,258,369 describes a method of treating patients suffering from chronic fatigue syndrome with dsRNA. This subset of patients had chronic cerebral dysfunction. MRI showed brain abnormalities. They developed a post-infectious immune dysfunction characterized by progressive mental deterioration, memory lapses, occasional seizures, and loss of higher mental abilities (i.e., chronic cerebral dysfunction). It was also discovered that the 2′-5′ oligoadenylate/RNase L pathway exists in these patients having chronic cerebral dysfunction. Further, natural killer (NK) cell function and phenotype were often unusual in this subset of patients. The majority of patients were infected by human herpes virus-6 (HHV-6).

Chronic fatigue syndrome patients were treated with AMPLIGEN® poly(I:C₁₂U) in a clinical study reported by Carter et al., Clinical Infectious Diseases, 18 (suppl. 1): S88-S95 (1994). The authors, however, did not disclose whether electrocardiography was used to assess the patients nor if there was any improvement in prolonged QT interval in them after treatment.

In none of these studies was dsRNA's effect on patients, who were selected for treatment of chronic fatigue syndrome, determined by electrocardiography to show a normalization of the QT interval in those who also exhibited a prolonged QT interval. This was a surprising result because this study was undertaken to determine if AMPLIGEN® poly(I:C₁₂U) increased the risk of proarrhythmic potential following lengthy treatment. No adverse effect was found. But an unexpected normalization of the QT interval was found in chronic fatigue syndrome patients

Therefore, we determined whether the prolonged QT interval of some chronic fatigue syndrome patients was improved by treatment with dsRNA. This subset of patients did not have to be selected for treatment based on whether their QT interval was prolonged or their concomitant use of medications resulting in a prolonged QT interval. Other advantages and improvements are described below or would be apparent from the disclosure herein.

### SUMMARY OF THE INVENTION

It is an objective of the invention to normalize the prolongation of QT interval or the induction of Torsades de Pointes (TdP) in a subset of human patients having chronic fatigue syndrome using at least one or more different double-stranded ribonucleic acids (dsRNA).

The effectiveness of treatment may be assessed by electrocardiography of an individual patient, or alternatively improved morbidity or mortality in a cohort. But the patient does not have to be selected for having an abnormal electrocardiogram or a cardiac disorder to be effectively treated using dsRNA. Alternatively, the patient may be selected for treatment by previously determining that the QT interval is prolonged, a medication that prolongs the QT interval is being consumed, a cardiac disorder that prolongs QT interval is diagnosed, or any combination thereof prior to administration of dsRNA. But it is emphasized that patients do not appear to suffer any increased risk of proarrythmic potential following lengthy exposure to dsRNA. The QT interval prolongation may be reduced from greater than or equal to 5 milliseconds.

In one aspect, at least one dsRNA is administered to a human patient in need of such treatment because of a diagnosis of chronic fatigue syndrome. Specifically configured or mismatched dsRNA is preferred, but other types of dsRNA may also be used. In particular, the specifically-configured dsRNA is a mismatched dsRNA. The dsRNA may be administered at a dosage of from about 10 to about 1200 mg/dose. This dosage may be administered once per week or month, or two or more doses per week or month. Each dose (e.g., from about 10 mg to about 1200 mg, from about 100 mg to about 800 mg, or from about 200 mg to about 400 mg) may be administered by intravenous infusion. Use of an effective amount of at least dsRNA may be continued until improvement in the severity and/or number of symptoms is achieved. The effective amount required to obtain such improvement may be identical to or higher than the amount required for maintenance of the effect(s).

In another aspect, a medicament is provided as a pharmaceutical composition containing one or more different dsRNA. In particular, the dsRNA may be specifically configured, or more preferably mismatched. Optional components of the composition include excipients and a vehicle (e.g., saline buffer) as a single dose or a multi-dose package (e.g., an injection vial or vials), and instructions for their use. Processes for making and using the pharmaceutical composition (medicament) are also provided. For example, one or more different dsRNA may be formulated at a concentration from about 1 mg/mL to about 5 mg/mL (e.g., 200 mg dissolved in 80 mL or 400 mg dissolved in 160 mL) in physiological phosphate-buffered saline and stored at from 2° C. to 8° C. in a refrigerator under aseptic conditions.

Further aspects of the invention will be apparent from the following description of specific embodiments and the appended claims, and generalizations thereto.

### BRIEF DESCRIPTION OF DRAWINGS

FIG. 1 shows the percentage of patients who had a decrease in the number of days of exposure to medications known to prolong QT interval by study (i.e., first or second) and randomized treatment assignment. For each comparison, results from the patients who received poly(I:C₁₂U) are represented by the left bar and results from the patients who received placebo are represented by the right bar. The first study has an odds ratio of 2.92 (95% confidence interval from 1.25 to 6.84); the second study has an odds ratio of 1.76 (95% confidence interval from 1.00 to 3.11).

FIG. 2 shows that patients randomized to receive poly(I:C₁₂U) were twice as likely to have a reduction in the use of concomitant medications known to prolong QT interval as compared to patients randomized to receive placebo. Results of the first and second studies were pooled. For each comparison, results from the patients who received poly(I:C₁₂U) are represented by the left bar and results from the patients who received placebo are represented by the right bar. Odds ratio: 2.02 (95% confidence interval from 1.27 to 3.21); Chi-square test: p=0.003.

### DESCRIPTION OF SPECIFIC EMBODIMENTS OF THE INVENTION

Chronic fatigue syndrome is diagnosed by use of criteria from the Center for Disease Control (CDC). See Fukuda et al., Annals of Internal Medicine, 12: 953-959 (1994). This fatigue is not improved by bed rest, and may be worsened by physical activity. Profound debilitation, which dramatically reduces a patient's ability to perform normal daily activities, may last for years. This inability of patients to exercise and their sedentary lifestyle is a significant risk factor for heart disease. Heart failure is a leading cause of death in chronic fatigue syndrome patients. Indeed, over 20% of the total deaths of patients were secondary to heart failure (Jason et al., Health Care for Women International, 27: 615-626, 2006), which is now known to induce QT interval prolongation most likely through upregulation of KCNE1.

No drugs are currently approved for the treatment of chronic fatigue syndrome. Accordingly, patients utilize many different drugs in a largely unsuccessful effort to relieve the chronically debilitating symptoms of chronic fatigue syndrome. Some of these drugs are known to prolong the QT interval. Many of the concomitant drugs utilized by chronic fatigue syndrome patients are known to prolong the QT interval or to induce Torsades de Pointes (TdP).

Drugs That Prolong QT Interval and/or Induce Torsades de Pointes

| Generic Name (Brand Name) | Drug Class/Clinical Usage |
|---|---|
| Amitriptyline (Elavil ®) | Tricyclic Antidepressant/depression |
| Azithromycin (Zithromax ®) | Antibiotic/bacterial infection |
| Ciprofloxacin (Cipro ®) | Antibiotic/bacterial infection |
| Clarithromycin (Biaxin ®) | Antibiotic/bacterial infection |
| Doxepin (Sinequan ®) | Tricyclic Antidepressant/depression |
| Fluconazole (Diflucan ®) | Anti-fungal/fungal infection |
| Fluoxetine (Prozac ®) | Anti-depressant/depression |
| Fluoxetine (Sarafem ®) | Anti-depressant/depression |
| Levofloxacin (Levaquin ®) | Antibiotic/bacterial infection |
| Salmeterol (Serevent ®) | Sympathomimetic/asthma, COPD |
| Sertraline (Zoloft ®) | Anti-depressant/depression |
| Sumatriptan (Imitrex ®) | Migraines/cluster headaches |
| Tizanidine (Zanaflex ®) | Muscle relaxant |
| Venlafaxine (Effexor ®) | Anti-depressant/depression |
| Zolmatriptan (Zomig ®) | Migraines |

Because of the importance of studying a potential proarrhythmic effect that might result from the administration of poly(I:C₁₂U) in the actual patient population being chronically exposed to this vast array of concomitant medications including many known to prolong QT interval, a QT/QTc study conducted in a chronic fatigue syndrome population is substantially superior to a QT/QTc study conducted in a normal healthy population of volunteers. Further, cumulative exposure to poly(I:C₁₂U) over 40 weeks in this severely debilitated population taking numerous QT interval prolonging drugs cannot be replicated in the normal healthy population. As shown in the example below, administration of dsRNA decreases concomitant medication use and improves QT interval prolongation.

The double-stranded ribonucleic acid (dsRNA) may be fully hybridized strands of poly(riboinosinic acid) and poly(ribocytidilic acid) (i.e., polyIC) or poly(riboadenylic acid) and poly(ribouracilic acid) (i.e., polyAU). The mismatched dsRNA may be of the general formula rIₙ·r(C₄₋₂₉U)ₙ, which is preferably rIₙ·r(C₁₂U)ₙ, in which r indicates ribonucleotides. It is preferred that n is an integer from about 40 to about 40,000. For example, a strand of poly(riboinosinic acid) may be partially hybridized to a strand of poly(ribocytosinic₄₋₂₉uracilic acid). Other mismatched dsRNA that may be used are based on copolynucleotides such as poly(CₘU) and poly(CₘG) in which m is an integer from about 4 to about 29 or analogs of a complex of poly(riboinosinic acid) and poly(ribocytidilic acid) formed by modifying the rI_{g}·rCₙ to incorporate unpaired bases (uracil or guanine) in the polyribocytidylate (rCₘ) strand. Alternatively, mismatched dsRNA may be derived from r(I)·r(C) dsRNA by modifying the ribosyl backbone of poly(riboinosinic acid) (rIₙ), e.g., by including 2′-O-methyl ribosyl residues. Of these mismatched dsRNA analogs of rIₙ·rCₙ, the preferred ones are of the general formula rIₙ·r(C₁₁₋₁₄U)ₙ or rIₙ·r(C₂₉,G)ₙ (see U.S. Pat. Nos. 4,024,222 and 4,130,641; which are incorporated by reference). The dsRNA described therein generally are suitable for use according to the present invention. See also U.S. Pat. No. 5,258,369. The dsRNA may be complexed with an RNA-stabilizing polymer such as polylysine, polylysine plus carboxymethylcellulose, polyarginine, polyarginine plus carboxymethylcellulose, or any combination thereof.

Other examples of mismatched dsRNA for use in the invention include:

r(I)·r(C₄, U),
r(I)·r(C₇, U),
r(I)·r(C₁₃, U),
r(I)·r(C₂₂, U),
r(I)·r(C₂₀, G) and
r(I)·r(C₂₉, G).

Mismatched dsRNA may also be modified at the molecule's ends to add a hinge(s) to prevent slippage of the base pairs, thereby conferring a specific bioactivity in specific solvents or aqueous environments which exist in human biological fluids.

dsRNA may be administered to a human patient by any local or systemic route known in the art including enteral (e.g., oral, feeding tube, enema), parenteral (e.g., subcutaneous, intravenous, intramuscular, intradermal, or intraperitoneal injection; buccal, sublingual, or transmucosal; inhalation or instillation intranasally or intratracheally), or topical (e.g., device such as a nebulizer for inhalation through the respiratory system, skin patch acting epicutaneously or transdermally, suppository acting in the rectum or vagina). dsRNA may be micronized by milling or grinding solid material, dissolved in a vehicle (e.g., sterile buffered saline or water) for injection or instillation (e.g., spray), topically applied, or encapsulated in a liposome or other carrier for targeted delivery. Preferred are carriers that target the dsRNA to the TLR3 receptor on antigen presenting cells and epithelium. For example, immature dendritic cells may be contacted in skin, mucosa, or lymphoid tissues. It will be appreciated that the preferred route may vary with the age, condition, or gender of the patient; the nature of disease, including the number and severity of symptoms; and the chosen active ingredient.

Formulations for administration (i.e., pharmaceutical compositions) may include aqueous solutions, syrups, elixirs, powders, granules, tablets, and capsules which typically contain conventional excipients such as binding agents, fillers, lubricants, disintegrants, wetting agents, suspending agents, emulsifying agents, preservatives, buffer salts, flavoring, coloring, and/or sweetening agents. It will be appreciated that the preferred formulation may vary with the age, condition, or gender of the patient; the nature of disease, including the number and severity of symptoms; and the chosen active ingredient.

The recommended dosage of dsRNA will depend on the clinical status of the patient and the physician's experience treating chronic fatigue syndrome. dsRNA may be dosed at from about 10 mg to about 1200 mg, from about 100 mg to about 800 mg, or from about 200 mg to about 400 mg in a patient (e.g., body mass of about 70 kg) on a schedule of once to thrice weekly (preferably twice weekly), albeit the dose amount and/or frequency may be varied by the physician in response to the patient's condition. Intravenous infusion of dsRNA dissolved in a physiological phosphate-buffered saline is preferred. Cells or tissues of the body that express TLR3 are preferred sites in the patient for delivering the nucleic acid, especially antigen presenting cells (e.g., dendritic cells and macrophages) and endothelium (e.g., endothelial cells of the respiratory and gastric systems). It will be appreciated that the preferred dosage may vary with the age, condition, or gender of the patient; the nature of disease, including the number and severity of symptoms; and the chosen active ingredient.

Dendritic cells which act as sentinel cells possess molecular surface structures that recognize pathogen-associated molecular patterns. These include a set of Toll-like receptors (TLRs) that specifically recognize double-stranded RNAs (i.e., Toll-like receptor 3 or TLR3). Poly(I:C₁₂U) is a selective agent for activation of TLR3, but other selective agents known in the art may be used. Patients with QT interval prolongation are at risk for increased morbidity and mortality. This abnormality may be normalized by using poly(I:C₁₂U) as a specific agonist of TLR3.

### Examples

A comprehensive analysis of QT interval assessments including QTc interval was performed in double-blinded, randomized, placebo-controlled studies of the administration of AMPLIGEN® poly(I:C₁₂U) for chronic fatigue syndrome (CFS) per ICH E14 (Clinical Evaluation of QT/QTc Interval Prolongation and Proarrhythmic Potential for Non-Anti-arrhythmic Drugs, October 2005, ICH). The clinical ECG database consists of 12-lead surface ECGs obtained during baseline (twice) and at week 20, 34 and 40 of each study. All ECGs were obtained by an experienced team headed by an exercise physiologist who traveled to each site and obtained the ECGs prior to administering the treadmill tolerance test.

Intra-patient average QTc (Bazzet) baseline values were examined relative to the E14 guidance using the categorical classification of QTc values. A recognizable subset of CFS patients were at increased risk of cardiovascular disease and cardiac-related events, consistent with the published literature. Concomitant medication by CFS patients has been well documented, and many of the concomitant medications used here are known to prolong the QT interval. To investigate the relationship of these specific medications on the QT interval, the mean QTc value was calculated for placebo patients who were taking no, one, and two or more concomitant medications known to prolong QT interval. There was a strong relationship between concomitant medication exposure and QT prolongation.

Within the placebo group, the change from baseline was not significantly different from zero for patients who were not exposed to concomitant medications known to prolong the QT interval. But the change from baseline was significantly different from zero (p<0.05) for placebo patients who were exposed to one or more concomitant medications known to prolong the QT interval.

To determine if the distribution of concomitant medications, known to prolong QT, differed significantly between the randomized treatment groups, the distribution of subjects was analyzed using a Fisher's exact test. Results of this examination revealed that the distributions were not significantly different between randomized treatment groups:

The arithmetic average QT values recorded at baseline and post-baseline were examined to determine if there was a difference based on concomitant medications known to prolong QT. The results of this examination clearly show that the exposure to concomitant medications known to prolong QT was associated with higher QT values, independent of the randomized treatment assignment, i.e., the relationship was consistent between the treatment groups.

A major risk factor for coronary artery disease, a leading cause of heart failure, is a sedentary life-style and lack of exercise which of course is the hallmark of CFS. The 5 msec increase in QT interval in the control (placebo) arm is explained in part by the long duration of the illness coupled with the disease-imposed sedentary life-style. In contrast, the poly(I:C₁₂U) arm had a medically and statistically significant increase in exercise ability.

The mean baseline QT and QTc intervals for the poly(I:C₁₂U) and placebo cohorts are not significantly different. Maximum on-study QT and QTc intervals (QTcB and QTcF) were determined for each subject and then categorized as being abnormal using the cutoff points for QT/QTc intervals >500, >480, and >450 msec or for QT/QTc increases from baseline of >30 and >60 msec.

There was no evidence of any QT or QTc parameter with more poly(I:C₁₂U) subjects with abnormal values when compared to the placebo. There was also no significant difference in the mean values recorded at baseline, based on the actual recorded QT values, or the corrected values using either the Bazett's or Fridericia's formula. Differences at baseline between the placebo and poly(I:C₁₂U) randomized treatment groups (Poly I:Poly C₁₂U-placebo) were −6.4, −3.0, and −4.2 for QT, Bazett's, and Fridericia, respectively. None of the differences were significant at the alpha=0.05 level.

The QT interval prolongation results provide a head-to-head comparison between subjects randomized to receive either poly(I:C₁₂U) or placebo. The increase in QT prolongation observed in the placebo group provides concurrent validation of the design, given a 5 ms post-treatment increase was observed in the placebo group of CFS subjects. The increase observed in the placebo patients can be directly attributable to the use of concomitant medications known to prolong QT coupled with a CFS disease imposed sedentary life-style known to be a risk factor for heart disease.

Within each of the well-controlled studies, the intra-patient aggregate number of days of exposure to medications with a known risk for QT interval prolongation was calculated for the initial four weeks of the study. The cumulative number of days of exposure to medications known to prolong QT interval was also calculated on an intra-patients basis using the medications recorded during the last four weeks of the study. For patients who withdrew prematurely, medications known to prolong QT interval taken during the last four weeks of study participation was calculated and used to assess the intra-patient change. The intra-patient change (last four weeks minus initial four weeks) was used to determine if the patient had a decrease in exposure to concomitant medications. The population for analysis included all patients who participated in the individual studies and could be evaluated. Patients who were never exposed to concomitant medications known to prolong QT interval were included in the analysis, reporting to the analysis as having experienced “no decrease in exposure” over the duration of the trial.

The fraction of patients who experienced a decrease was compared between randomized treatment groups using a chi-square test. The odds ratio was calculated including the 95% confidence interval. The pooled study results consider the last four weeks of exposure within each study, which were conducted for a total of 24 and 40 weeks.

### First Clinical Study

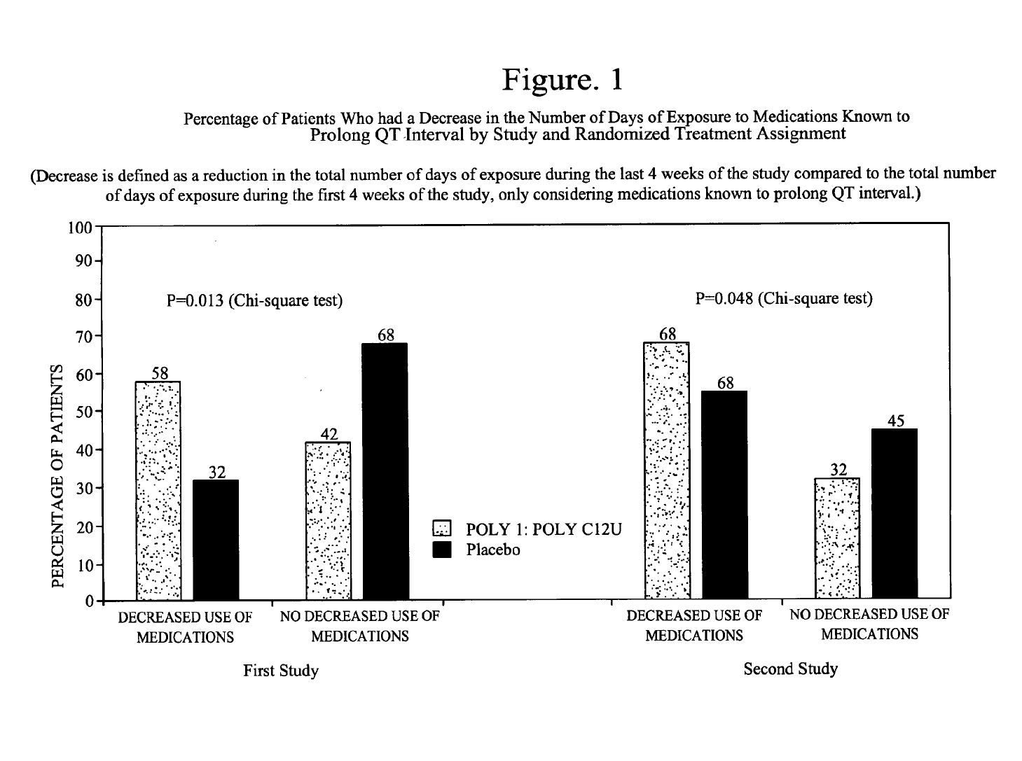

In this double-blinded, randomized, placebo-controlled study, there were 92 patients who constituted the evaluable patient population. Twenty-six of the 45 patients (57.8%) of the patients randomized to receive poly(I:C₁₂U) experienced a reduction in exposure to concomitant medications with a known risk of prolonging QT interval, compared to 15 of the 47 patients (31.9%) randomized to receive placebo. Comparing the proportion of patients between randomized treatment assignment and reduced exposure revealed a significant difference in favor of poly(I:C₁₂U) (p=0.013). Patients randomized to receive poly(I:C₁₂U) were approximately three times more likely to have reduced exposure to medications known to prolong QT interval as compared to patients randomized to receive placebo (FIG. 1).

### Second Clinical Study

In this double-blind, randomized, placebo controlled study, there were 208 patients who constituted the evaluable patient population. Sixty-eight of the 100 patients (68.0%) of the patients randomized to receive poly(I:C₁₂U) experienced a reduction in exposure to concomitant medications with a known risk of prolonging QT interval, compared to 59 of the 108 patients (54.6%) randomized to receive placebo. Comparing the proportion of patients between randomized treatment assignment and reduced exposure revealed a significant difference in favor of poly(I:C₁₂U)=0.048). Patients randomized to receive poly(I:C₁₂U) were one and three-quarters times more likely to have reduced exposure to medications known to prolong QT interval as compared to patients randomized to receive placebo (FIG. 1).

### Pooled Study Results

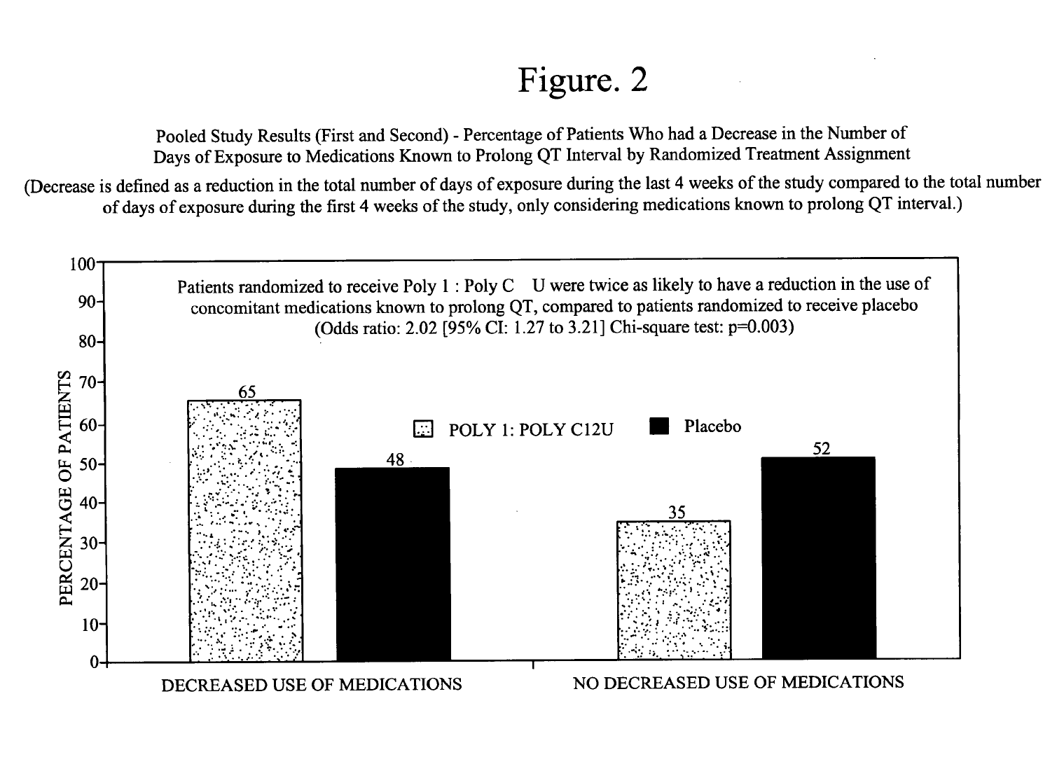

In the two'double-blinded, randomized, and placebo-controlled studies, there were a total of 300 patients who constituted the evaluable patient population. Ninety-four of the 145 patients (64.8%) of the patients randomized to receive poly(I:C₁₂U) experienced a reduction in exposure to concomitant medications with a known risk of prolonging QT interval, compared to 74 of the 155 patients (47.7%) randomized to receive placebo. Comparing the fraction of patients between randomized treatment assignment and reduced exposure revealed a significant difference in favor of poly(I:C₁₂U) (p=0.003). Patients randomized to receive poly(I:C₁₂U) were twice as likely to have reduced exposure to medications known to prolong QT interval as compared to patients randomized to receive placebo (FIG. 2).

### CONCLUSION

Within each of the two studies, a significant reduction in cumulative duration of exposure was observed. These results from two independent clinical studies suggest that the therapeutic benefit of poly(I:C₁₂U) allows patients to reduce their dependence on concomitant medications for the symptoms and morbidity associated with chronic fatigue syndrome. Specifically, reducing the exposure to medications with a known risk associated with QT interval prolongation is an important clinical endpoint that underscores the seriousness of chronic fatigue syndrome, and the lack of effective treatments for this disenfranchised patient population.

Patents, patent applications, books, and other publications cited herein are incorporated by reference in their entirety.

In stating a numerical range, it should be understood that all values within the range are also described (e.g., one to ten also includes every integer value between one and ten as well as all intermediate ranges such as two to ten, one to five, and three to eight). The term “about” may refer to the statistical uncertainty associated with a measurement or the variability in a numerical quantity which a person skilled in the art would understand does not affect operation of the invention or its patentability. All modifications and substitutions that come within the meaning of the claims and the range of their legal equivalents are to be embraced within their scope. A claim which recites “comprising” allows the inclusion of other elements to be within the scope of the claim; the invention is also described by such claims reciting the transitional phrases “consisting essentially of” (i.e., allowing the inclusion of other elements to be within the scope of the claim if they do not materially affect operation of the invention) or “consisting of” (i.e., allowing only the elements listed in the claim other than impurities or inconsequential activities which are ordinarily associated with the invention) instead of the “comprising” term. Any of these three transitions can be used to claim the invention.

It should be understood that an element described in this specification should not be construed as a limitation of the claimed invention unless it is explicitly recited in the claims. Thus, the granted claims are the basis for determining the scope of legal protection instead of a limitation from the specification which is read into the claims. In contradistinction, the prior art is explicitly excluded from the invention to the extent of specific embodiments that would anticipate the claimed invention or destroy novelty.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of individual elements disclosed herein are considered to be aspects of the invention. Similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

1. A method of treating a patient having chronic fatigue syndrome, said method comprising administration to the patient of at least double-stranded ribonucleic acid (dsRNA) in a therapeutic amount to decrease a prolonged QT interval of the patient.

2. The method according to claim 1 further comprising evaluating the patient by electrocardiography at least before, during, or after treatment.

3. The method according to claim 1, wherein the patient reduces exposure to one or more drugs associated with prolongation of QT interval at least during or after treatment.

4. The method according to claim 1, wherein the dsRNA is mismatched dsRNA.

5. The method according to claim 4, wherein the mismatched dsRNA comprises poly(I:C₄₋₂₉U).

6. The method according to claim 4, wherein the mismatched dsRNA comprises

7. The method according to claim 4, wherein the mismatched dsRNA comprises poly(I:C₁₂U).

8. The method according to claim 1, wherein at least dsRNA in a therapeutic amount is infused intravenously.

9. The method according to claim 1, wherein at least dsRNA in a therapeutic amount is injected intradermally, subcutaneously, or intramuscularly; inhaled intranasally or intratracheal̂; or applied intranasally, intratracheally, oropharyngeal̂, or sublingually.

10. Use of at least double-stranded ribonucleic acid (dsRNA) in a therapeutic amount to treat a patient having chronic fatigue syndrome and prolonged QT interval.

11. Use of at least double-stranded ribonucleic acid (dsRNA) in a therapeutic amount to manufacture a medicament used to treat a patient having chronic fatigue syndrome and prolonged QT interval.
